(19) United States
(12) Patent Application Publication
(10) Pub. No.: US 2007/0105220 A1
(43) Pub. Date: May 10, 2007

(54) Erythrocytic cells and method for loading solutes

(75) Inventors: John H. Crowe, Davis, CA (US); Fern Tablin, Davis, CA (US); Nelly Tsvetkova, Davis, CA (US); Zsolt Torok, Davis, CA (US); Gyana R. Satpathy, Davis, CA (US); Denis Dwyre, Iowa City, IA (US); Rachna Bali, West Sacramento, CA (US); Mitali Banerjee, Davis, CA (US); Azadeh Kheirolomoom, Sacramento, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Regents of the University of California, Oakland, CA (US)

(21) Appl. No.: 10/567,595

(22) PCT Filed: Aug. 6, 2004
(86) PCT No.: PCT/US04/25395

§ 371 Date: Dec. 14, 2006

Related U.S. Application Data
(63) Continuation-in-part of application No. 10/635,396, filed on Aug. 6, 2003, now abandoned.
(63) Continuation-in-part of application No. 10/635,795, filed on Aug. 6, 2003, now abandoned.
(63) Continuation-in-part of application No. 10/635,754, filed on Aug. 6, 2003, now abandoned.
(63) Continuation-in-part of application No. 10/724,372, filed on Nov. 28, 2003, now abandoned.
Application No. 10/724,372 is a continuation-in-part of application No. 10/635,795, filed on Aug. 6, 2003, now abandoned.

(51) Int. Cl. A01N 1/02; C12N 5/08

(52) U.S. Cl. 435/372; 424/600; 435/2

(57) ABSTRACT

A dehydrated composition is provided that includes freeze-dried erythrocytic cells. The invention further provides methods for loading a solute into a cell comprising disposing a cell in a solution having a solute concentration of sufficient magnitude to produce hyperosmotic pressure on the cell for transferring a solute from the solution into cell.

### CROSS-REFERENCES TO RELATED APPLICATIONS

This patent application claims the benefit of U.S. patent application Ser. No. 10/635,754, filed Aug. 6, 2003, U.S. patent application Ser. No. 10/635,396, filed Aug. 6, 2003, U.S. patent application Ser. No. 10/635,795, filed Aug. 6, 2003, and U.S. patent application Ser. No. 10/724,372, filed Nov. 28, 2003. All of these patent applications are incorporated herein by reference.

### STATEMENT AS TO RIGHTS TO INVENTIONS MADE UNDER FEDERALLY SPONSORED RESEARCH OR DEVELOPMENT

Embodiments of this invention were made with Government support under Grant No. N66001-00-C-8048, awarded by the Department of Defense Advanced Research Projects Agency (DARPA). Further embodiments of this invention were made with Government support under Grant Nos. HL57810 and HL61204, awarded by the National Institutes of Health. The Government has certain rights in this invention.

### REFERENCE TO A “SEQUENCE LISTING,” A TABLE, OR A COMPUTER PROGRAM LISTING APPENDIX SUBMITTED ON A COMPACT DISK.

Not applicable.

### FIELD OF THE INVENTION

Embodiments of the present invention generally broadly relate to living mammalian cells. More specifically, embodiments of the present invention generally provide for the preservation and survival of erythrocytes.

Embodiments of the present invention also generally broadly relate to the therapeutic uses of erythrocytic cells, such as loading erythrocytic cells with solutes and in preparing dried compositions that can be re-hydrated at the time of application. In other aspects, the invention relates to reducing hemolysis and eliminating osmotically-fragile red blood cells.

The compositions and methods for embodiments of the present invention are useful in many applications, such as in medicine, pharmaceuticals, biotechnology, and agriculture, and including transfusion therapy, as hemostasis aids and for drug delivery.

### BACKGROUND OF THE INVENTION

Erythrocytes are one of the cellular components of the blood. In their normal mature form, erythrocytes are non-nucleated biconcave disks adapted by their morphology and hemoglobin content to transport oxygen. The mature form of erythrocytes are often referred to as “red blood cells.”

Erythrocytes are useful in a variety of clinical and laboratory assays, and have critical medical uses. Unfortunately, it is difficult to freeze or otherwise preserve erythrocytes. Erythrocytes can be frozen by placing them in glycerol, but the glycerol must be removed before the cells can be infused into a patient or used in assays. It would be desirable to have other means to preserve erythrocytes.

Trehalose has been found to be suitable in the cryopreservation of some kinds of cells. Trehalose is a disaccharide found at high concentrations in a wide variety of organisms that are capable of surviving almost complete dehydration. Trehalose has been shown to stabilize membranes, proteins, and certain cells during freezing and drying in vitro. Unfortunately, erythrocytes lack effective mechanisms for endocytosis, and it is very difficult to load erythrocytes with solutes unless the erythrocytes has a transporter specific for that solute. Erythrocytes do not have a transporter for trehalose. Accordingly, it would be desirable to have a convenient method for loading erythrocytes with trehalose or other solutes for which the cells lack an active transporter.

### BRIEF SUMMARY OF THE INVENTION

In one set of embodiments, the invention provides methods for loading a solute into an erythrocytic cell, comprising disposing an erythrocytic cell in a solution having a solute concentration of sufficient magnitude to produce hyperosmotic pressure on the cell, thereby transferring a solute from the solution into the cell. Preferably, the solute is present in said solution in a concentration of between 700 and 1000 mM. The solute can be a disaccharide. In some embodiments, the disaccharide is trehalose. The loading solution can further comprise a potassium salt. The potassium salt can be, for example, potassium phosphate. The loading solution can further comprise α-crystallin. The solution can further comprise a strong reducing agent. In some embodiments, the strong reducing agent is ascorbic acid. In some embodiments, the solution comprises a disaccharide, α-crystallin, ascorbic acid, and a potassium salt. In some embodiments, the loading is conducted at a temperature of between 25 and 40° C. The loading is preferably conducted at a temperature of between 30 and 40° C. and is most preferably conducted at a temperature of about 37° C.

In another set of embodiments, the invention provides an erythrocyte loaded with from 10 mM to 50 mM trehalose. The erythrocyte can further comprise ascorbic acid. The erythrocyte can further comprise α-crystallin.

In another set of embodiments, the invention provides methods for separating fragile or damaged cells from a population of erythrocytes. The methods comprise contacting the population with a first solution which is hyperosmotic with respect to a solute, loading a solute into the erythrocytes, removing the erythrocytes from the hyperosmotic solution, contacting the erythrocytes with a second solution which is mildly hypoosmotic in comparison to the hyperosmotic solution, thereby lysing fragile or damaged cells, and separating said fragile or damaged cells from the population. In some embodiments, the separation is by centrifugation.

In another set of embodiments, the invention provides methods for freeze-drying erythrocytes comprising lowering the hematocrit of said erythrocytes to between 2 and 5%.

In another set of embodiments, the invention provides methods for freeze-drying erythrocytes, comprising drying said erythrocytes in the presence of liposomes. In some embodiments, the liposomes are composed primarily of unsaturated lipids.

In some embodiments, the invention provides methods for freeze-drying erythrocytes, comprising freeze-drying said erythrocytes in the presence of 200-300 mOsm of potassium salts. In these methods, the erythrocytes may be present in a hematocrit of up to 15%.

In another set of embodiments, the invention provides buffers for freeze drying erythrocytes. The buffers may comprise liposomes. The liposomes are preferably composed primarily of unsaturated lipids. The buffers may comprise ascorbic acid.

In another set of embodiments, the invention provides buffers for rehydrating dried erythrocytes. The buffers may comprise methylene blue. The buffers may comprise transition metal ions. The transition metal ions may be, for example, zinc, copper, magnesium, gold or nickel. The buffers may further comprise ascorbic acid. In some embodiments, the invention provides solutions for rehydrating dried erythrocytes which comprise methylene blue, ascorbic acid, and transition metal ions.

The invention further provides methods for rehydrating dried erythrocytes. In some embodimens, the methods comprise contacting dried erythrocytes with a solution comprising methylene blue. In some embodiments, the methods comprise contacting dried erythrocytes with a solution comprising transition metal ions. In some embodiments, the methods comprise contacting dried erythrocytes with a solution comprising ascorbic acid. In some embodiments, the methods comprise contacting dried erythrocytes with a solution comprising liposomes, methylene blue, and transition metal ions.

### BRIEF DESCRIPTION OF THE DRAWINGS

FIG. 1 graphically illustrates intracellular trehalose concentration in erythrocytic cells as a function of extracellular trehalose concentration at respective temperatures of 4° C. and 37° C.;

FIG. 2 graphically illustrates the fragility index of erythrocytic cells incubated overnight at respective temperatures of 4° C. and 37° C. in the presence of and as a function of increasing intracellular trehalose concentrations;

FIG. 3 graphically illustrates trehalose uptake (i.e., intracellular trehalose mM) and hemolysis (i.e., % hemolysis) as a function of incubation temperature (° C.); and

FIG. 4 graphically illustrates intracellular trehalose (mM) as a function of the osmolarity of the washing buffer.

### DETAILED DESCRIPTION OF THE INVENTION

Red blood cells lack effective endocytoic mechanisms, which limit the ability to load them with solutes. We have now discovered methods for loading erythrocytes, including red blood cells, with solutes, such as trehalose. Additionally, we have discovered methods of improving the survival of such cells when dried and then rehydrated and for removing fragile and damaged cells from the erythrocyte population. Thus, the methods and compositions of the invention permit improved methods for providing erythrocytes that can be stored and rehydrated when needed.

We have surprisingly discovered that solutes for which erythrocytes lack active transporters can be loaded into erythrocytes by incubating the erythrocytes in hyperosmotic loading buffers. The solute is preferably in the loading buffer at a concentration of about 700 to about 1000 mM, with 800 mM being particularly preferred. Trehalose is a particularly useful solute with which to load erythrocytes because it tends to stabilize proteins, and therefore tends to help the erythrocytes survive a variety of in vitro and in vivo conditions. Our tests have noted no changes in erythrocytes loaded with trehalose which appear to affect their activity.

Surprisingly, we have also discovered that fragile or damaged cells can be removed from the population of erythrocytes loaded with solutes, such as trehalose, by first loading the population using a hyperosmotic loading solution, as described above, and then contacting the population of cells with a solution, such as a washing solution or buffer, that is mildly hypoosmotic compared to the loading buffer (a washing buffer that was severely hypoosmotic compared to the loading buffer would cause the most or all of the cells to lyse, and is of course not preferred). Fragile cells are considered to be aged cells at the end of their useful life and they, and damaged cells are more likely to lyse during blood processing and transfusion. This is disadvantageous lysed cells can release free hemoglobin into the blood, and free hemoglobin can cause kidney damage. Further, the lysed cells can reseal, forming empty, non-functional cells known as “ghosts”, or can fuse with functional cells, interfering with their activity. Thus, it is useful to remove fragile and damaged erythrocytes from the erythrocytic population before, for example, infusing the erythrocytes into a patient.

The methods described herein cause selective lysing of the fragile or damaged erythrocytes. As noted, the cells loaded by the hyperosmotic loading procedure described above and herein are contacted with a solution that is mildly hyposmotic compared to the loading buffer. It is desirable that this second solution, which may be conveniently referred to as a washing buffer, has an osmolarity less than the osmolarity of the loading buffer, but not more than 200 mOsm less, preferably not more than 150 mOsm less, more preferably not more than about 100 mOsm less, still more preferably about or at 50 mOsm. The washing buffer can have as little as 25 mOsm less osmolarity than the loading buffer. The difference in the osmolarity “pops” the fragile and damaged cells. Five minutes to one hour contacting with the washing buffer is preferred. There is an inverse ratio between the amount of time and the relative hypoosmolarity, so as the washing buffer is more hypoosmolar, the time the cells are washed should be decreased to avoid lysing normal cells. With a preferred osmolarity of 50 mOsm difference between the loading and wash buffers, the cells should be washed for 5 to 15 minutes.

We have further found that the hyperosmotic loading of solutes such as trehalose can be used to prepare the erythrocytes for preservation by freezing. Optionally, the cells are also loaded with any of a number of compounds known in the art to aid in preserving erythrocyte function, such as adenine, inosine, sorbitol, or ascorbic acid, or mixtures thereof In a preferred embodiment, we have found good results using the commercially available product Adsol® (Cytosol Laboratories Inc., Braintree, Mass.). Adsol® comprises 11 mM glucose, 2mM adenine, 154 mM NaCl, 41 mM mannitol, with a total milliosmolarity of 462.) Preferably, the Adsol® is present at about 25 milli Osmole (mOsm) to about 400 mOsm, more preferably about 50 mOsm to about 300 mOsm, still more preferably about 50 mOsm to about 200 mOsm, even more preferably about 75 mOsm to about 150 mOsm, and most preferably about 100 mOsm.

The loading solution preferably further comprises a potassium salt, such as potassium phosphate, potassium chloride, potassium citrate, or a combination of these. In some embodiments, the concentration of potassium salt is about 1 mM to 30 mM, more preferably about 1 mM to about 25 mM, still more preferably about 2 mM to about 20 mM, yet more preferably about 3 mM to about 15 mM, even more preferably about 4 mM to about 12 mM, still more preferably about 5 mM to about 10 mM, even more preferably about 6 mM to about 8 mM, even more preferably about 6 mM to about 7 mM, and most preferably about 6.7 mM. The pH of the solution is desirably about pH 6 to about pH 8, more preferably between about pH 6.5 to about 7.8, still more preferably about 6.7 to about 7.5, and most preferably about pH 7.2.

Optionally, the hyper osmotic loading solution further comprises α-crystallin. As is known in the art, α-crystallin is a molecular chaperone and lens structural protein that protects soluble enzymes against heat-induced aggregation and inactivation by a variety of molecules. See, e.g., Derham et al., Eur. J. Biochem, 270(12): 2605-2611 (2003). The concentration of α-crystallin in the hyperosmotic solution is preferably about 0.01 mg/mL to about 100 mg/mL. More preferably, it is about 0.1 mg/mL to about 10 mg/mL. In some embodiments, it is about 0.5 mg/mL to about 5 mg/mL. In others, it is about 0.5 mg/mL to about 3 mg/mL, and in others about 0.5 mg mg/mL to about 2 mg/mL. We have found that a concentration of about 1 mg/mL increases cell survival by about 10%, and that concentration is most preferred.

We have further found that the uptake of trehalose or other solutes is enhanced by incubating the erythrocytes at temperatures between about 30° C. to about 40° C. Most preferably, the cells are incubated at temperatures approximating physiological temperatures for the species from which the cells originated. For human cells, it is preferable that the cells be incubated at or around 37° C.

We have further found that the cells should generally be incubated for several hours, but that the effect peaks after an interval; thus, an overlong incubation is not helpful and may be disadvantageous. The incubation should be from about 3 to 14 hours, with 4 to 12 hours being preferred, 5 to 10 hours being more preferred, 6 to 9 hours being still more preferred, about 6 to about 8 being preferable, and 7 hours being the best choice for maximal loading and use of time.

If the removal of fragile and damaged cells is desired once the population is loaded with the solute of choice, the population is contacted with a washing buffer, as described above. The erythrocyte population is then preferably centrifuged to separate the intact loaded cells from the lysed fragile or damaged cells. Typically, the population is centrifuged at a speed and for a time sufficient to cause separation of the intact cells from the erythrocyte “ghosts”. Typically, the cells are spun at 1500 rpm for 5 minutes. The supernatant, containing the free hemoglobin and erythrocyte “ghosts” is removed. The pelleted erythrocytes are resuspended in a solution preferably containing one or more erythrocyte protecting substances, such as adenine. Preferably, the resuspension solution is Rejuvesol® (Cytosol Laboratories Inc., Braintree, Mass.). Rejuvesol® was approved in 1997 by the U.S. Food and Drug Administration for rejuvenating the activity of red blood cells stored in Adsol®, and comprises 100 mM sodium pyruvate, 100 mM inosine, 70.4 mM Na₂HPO₄, 29 mM NaH₂PO₄, and 5 mM adenine. Conversely, one or more of these or other known erythrocyte-protectants can be used in the solution in place of the Rejuvesol®. The cells are typically incubated in this solution for about 10 to 100 minutes, preferably about 30 minutes, at a hematocrit between 10 and 70%, more preferably 20 to about 50%, and still more preferably about 30%. The cells are then centrifuged gently (for example, at 1000 rpm for 5 minutes) and the supernatant removed.

The loaded cells can be dried, preferably by freeze-drying (lyophilization). Freeze drying is usually preferred in part because it is the most economical for drying large volumes of materials.

We have found that the presence of transition metal ions in the rehydration buffer helps in reducing the hemolysis of erythrocytes during rehydration. Preferably, the transition metal ions are one or more of the following: zinc, nickel, copper, or magnesium. Zinc is particularly preferred.

The process of freeze drying, in particular, can change the form of some or all of the hemoglobin present in some or all of the cells to methemoglobin, thereby affecting the ability of the cells to transport oxygen. It would be desirable to reduce the change of hemoglobin to methemoglobin, and to convert any hemoglobin that has changed to methemoglobin back to hemoglobin.

We have now discovered that the change of hemoglobin to methemoglobin can be reduced by loading the cells with a biologically acceptable, strong reducing agent prior to the drying process. Conveniently, the strong reducing agent can be in the hyperosmotic loading buffer described above. Optionally, the strong reducing agent is also present in the drying buffer, such as a freeze drying buffer, in the rehydration buffer, or in both. Surprisingly, the presence of the strong reducing agent also increases the percentage of the cells that survive drying and rehydration by approximately 5%, as well as improving their oxygen transport. Preferably, the strong reducing agent is ascorbic acid.

We have now further discovered that the hemoglobin that has converted to methemoglobin can be reconverted to hemoglobin by the use of methylene blue. Preferably, the methylene blue is present in the rehydration buffer. The methylene blue can be used in combination with ascorbic acid, as described above, or by itself. Preferably, the buffer contains 0.1 to about 100 μM methylene blue. In some embodiments, the methylene blue is present in about 0.5 to about 75 μM, and in others about 1 to about 60 μM. In some embodiments, the methylene blue is present in about 2 to about 50 μM, and in others about 4 to about 40 μM. In some embodiments, the methylene blue is present in about 5 to about 35 μM, and in others about 6 to about 30 μM. In some embodiments, the methylene blue is present in about 7 to about 25 μM, and in others about 8 to about 20 μM. Preferably, about 10 μM methylene blue is present.

Surprisingly, we have also discovered that the presence of liposomes in the drying buffer, preferably a freeze-drying buffer, sharply improves the survival of erythrocytes dried in the buffer. The lipids comprising the liposomes can be saturated, unsaturated, or both. We have had better survival when the liposomes are primarily unsaturated, so liposomes of unsaturated lipids are preferred. The liposomes can be present at about 2 to 200 mg/mL, more preferably about 4 to about 125 mg/mL, still more preferably about 5 to 100 mg/mL. In some embodiments, the liposomes are present at about 7 to about 80 mg/mL, and in others, they are present at about 8 to about 60 mg/mL. In still other embodiments, the liposomes are present at about 10 to about 50 mg/mL, and in others, they are present at about 15 to about 35 mg/mL. In some embodiments, the liposomes are present at about 16 to about 30 mg/mL, and in others, they are present at about 18 to about 25 mg/mL. Preferably, the liposomes are present at about 20 mg/mL.

Also surprisingly, we have discovered that the cell concentration, or “hematocrit,” during the drying process makes a difference in the survival of dried erythrocytes upon rehydration. Surprisingly, lower hematocrits usually increase cell survival upon rehydration. Hematocrits between about 2-5%, and especially between about 3-5%, during drying strike a reasonable balance between increased survival and having a reasonable number of cells per volume, and are preferred. We have also discovered, however, that use of freeze drying buffers in which the concentration of potassium salt or salts is increased to between 200 and 300 mOsm, and without trehalose, permits increasing the hematocrit up to 15% without reducing cell viability. This is advantageous since it permits drying more cells per volume of liquid and is accordingly preferred.

Rehydrated erythrocytes preferably are diluted to isotonic conditions. Conveniently, the cells are pelleted by centrifugation, for example, at 11,000 rpm for 4 min., and resuspended in a mixture of 50% Dulbecco's phosphate-buffered saline (“DPBS”) and 50% rehydration buffer. Further, the cells are recentrifuged at 8,000 rpm for 4 min and resuspended in 75% DPBS and 25% rehydration buffer. Finally, they are centrifuged at 5000 for 3 minutes and resuspended in 100% DPBS.

The terms “erythrocyte” and “erythrocytic cell” designate one of the cellular components of the blood. As defined by Dorland's Medical Dictionary (on the World Wide Web at merckmedicus.com/pp/us/hcp/thcp_dorlands_content. jsp?pg=/ppdocs/us/common/dorlands/dorlaiid/dmd-e-025.htm), in their normal mature form, erythrocytes are non-nucleated biconcave disks adapted by their morphology and hemoglobin content to transport oxygen. The mature form of erythrocytes are often referred to as “red blood cells,” or “RBCs”. The number of RBCs usually is far in excess of the immature erythrocytic cells, and therefore a population of erythrocytes is generally conveniently referred to as red blood cells. As used herein, “erythrocyte” or “erythrocytic cell” refers to any form of erythrocyte, including immature forms, unless otherwise indicated or required by context. References herein to “cells” refers to erythrocytic cells unless otherwise indicated or required by context. Mammalian, and in particular, human, erythrocytes are preferred. Suitable mammalian species for providing erythrocytic cells for use in the invention include, by way of example only, human, equine, canine, feline, and bovine species.

Broadly, the preparation of solute-loaded cells in accordance with embodiments of the invention comprises the steps of loading one or more cells with a solute by placing one or more cells in a solution having a solute concentration of sufficient magnitude to produce hyperosmotic pressure on the cell for transferring the solute from the solution into the cell. For increasing the transfer or uptake of the solute from the solute solution, the solute solution temperature or incubation temperature has a temperature above about 25° C., more preferably above 30° C. and at or below 40° C., such as from about 30° C. to about 40° C. In another embodiment of the invention, a solute solution (e.g., trehalose solution) has a solute (e.g., trehalose) concentration of at least about 25%, preferably at least about 50%, greater than the intracellular osmolarity of the cells for loading the solute into the cells. For various embodiments of the invention, a solute solution has a solute concentration ranging from about 25% to at least about 1000% greater than the intracellular osmolarity of the cell. In some embodiments, the concentration of the solute is about 725 to about 950 mM; in others it is about 750 mM to about 925 mM, in still others, it is about 750 mM to about 900 mM. In some embodiments, the concentration of the solute is about 750 to about 875 mM; in others it is about 750 mM to about 850 mM, in still others, it is about 775 MM to about 850 mM. In some embodiments, the concentration of the solute is about 775 to about 840 mM; in others it is about 775 mM to about 830 mM, in still others, it is about 780 mM to about 825 mM. In some embodiments, the concentration of the solute is about 785 to about 820 mM; in others it is about 785 mM to about 815 mM, in still others, it is about 790 mM to about 810 mM. It is most preferred that the concentration of the solute be about 800 mM.

We determined the fragility index of erythrocytic cells incubated overnight at temperatures of 4° C. and 37° C., respectively, in the presence of and as a function of increasing intracellular trehalose concentrations. The osmotic fragility index was generated by the extent of hemolysis as a function of the NaCl concentration as a test for investigating the effects of hyperosmotic treatment rendering erythrocytic cells more sensitive to change in intracellular osmolarity. NaCl was loaded into erythrocytic cells from a 100 mOsm PBS buffer at loading 100 mOsm PBS buffer temperatures of 4° C. and 37° C. for extracellular trehalose concentrations of 0 mM (control cells), 250 mM, 500 mM, 600 mM, 700 mM, 800 mM and 1000 mM. The erythrocytic cells that had been loaded in trehalose solutions (between 250 mM and 1000 mM) in 100 mOsm PBS were suspended in increasing concentrations of NaCl (between 50 and 600 mOsm NaCl). The percent hemolysis measured after resuspending the loaded cells in NaCl represents the fragility index. The data show that the erythrocytic cells were stable osmotically in trehalose media with concentrations between 250 mM and 800 mM trehalose at both 37° C. and 4° C. In 1000 mM trehalose at 37° C., there is a high increase in the fragility index suggesting that the cells were unstable in this medium (1000 mM trehalose in 100 mOsm PBS). At moderate intracellular concentrations of trehalose, osmotic fragility as measured by a standard assay was not severely altered. Thus, erythrocytic cells may be loaded with trehalose concentrations up to about 900 mM (i.e., a trehalose concentration between 800 mM and 1000 mM).

The method may additionally comprise preventing a decrease in a loading gradient and/or a loading efficiency gradient in the loading of the solute into the cells. Preventing a decrease in a loading efficiency gradient in the loading of the solute into the cells comprises maintaining a positive gradient of loading efficiency (e.g., in %) to concentration (e.g., in mM) of the solute in the solute solution. Preventing a decrease in a loading gradient in the loading of the oligosaccharide into the cells comprises maintaining a concentration of the solute in the solute solution below a certain concentration (e.g., below a concentration ranging from about 35 mM to about 65 mM, more particularly below from about 40 mM to about 60 mM, or below from about 45 mM to about 55 mM, such as below about 50 mM); and/or maintaining a positive gradient of concentration of solute loaded into the cells to concentration of the solute in the solute solution.

The solute solution may be any suitable physiologically acceptable solution in an amount and under conditions effective to cause uptake or “introduction” of the solute from the solute solution into the cells. A physiologically acceptable solution is a suitable solute-loading buffer, such as any of the buffers stated in the previously mentioned related patent applications, all having been incorporated herein by reference thereto.

The solute is preferably a carbohydrate (e.g., an oligosaacharide) selected from the following groups of carbohydrates: a monosaccharide (e.g., bioses, trioses, tetroses, pentoses, hexoses, heptoses, etc), a disaccharide (e.g., lactose, maltose, sucrose, melibiose, trehalose, etc), a trisaccharide (e.g., raffinose, melezitose, etc), or tetrasaccharides (e.g., lupeose, stachyose, etc), and a polysaccharide (e.g., dextrins, starch groups, cellulose groups, etc). More preferably, the solute is a disaccharide, with trehalose being the preferred, particularly since it has been discovered that trehalose does not degrade or reduce in complexity upon being loaded. Thus, in the practice of various embodiments of the invention, trehalose is transferred from a solution into the cells without degradation of the trehalose.

An extracellular medium of about 280-320 mOsm is considered iso-osmotic for erythrocytic cells with regard to the amount of permeable solutes in the cytoplasm. Any increase of the amount of solutes in the extracelluar medium creates an osmotic shock, ranging from a mild shock at about 350 mM trehalose to a strong shock at about 4200 mM trehalose, and a leakage of water which would reversibly reduce the cell volume. However, small molecular weight solutes, such as trehalose, in an extracellular medium in a concentration higher than about 320 mM, can pass through the membrane of a cell using a diffusion vector. It has been discovered that an extracellular concentration of trehalose higher than about 450 mM (or mOsm), which is about 50% greater than an intracellular milliosmolarity, will produce an osmotic shock that will result in trehalose uptake. Increasing the extracellular trehalose concentration leads to even higher osmotic shock and higher trehalose uptake.

Molarity, or millimolarity, mM, is the number of moles (or millimoles) of a solute per liter of solution and is a measure of the concentration. Osmolarity (Osm), or milliosmolarity (mOsm), is a count of the number of dissolved particles per liter of solution and is a measure of the osmotic pressure exerted by solutes. Biological membranes, such as cell membranes, can be semi-permeable because they allow water and some small molecules to pass, but block the passage of proteins or macromolecules. Since the osmolarity of a solution is equal to the molarity times the number of particles per molecule, 600 mM trehalose is equal to 600 mOsm trehalose because trehalose does not dissociate in water. However, with respect to compounds that dissociate in water, such as NaCl, 1 mM NaCl is equal to 2 mOsm NaCl because it has two particles. Similarly, 100 mM NaCl is equal to 200 mOsm NaCl. Thus, for a 300 mOsm PBS buffer (100 μM NaCl, 9.4 mM Na₂HPO₄, 0.6 mm KH2PO4, pH 7.4), 300 mOsm refers to all of the osmotically active particles in the PBS solution, with 200 mOsm of the 300 mOsm stemming from NaCl. A suitable PBS buffer for various embodiments of the present invention comprises 154 mM NaCl, 1.06 mM Na₂HPO₄, 5.6 mm KH₂PO₄, pH 7.4.

Optionally, the cells can be washed in a washing buffer after loading. If the desire is to remove fragile and damaged cells, it will be desirable to have an osmotic gradient between the loading buffer and the wash buffer to encourage the fragile and the damaged cells to lyse. If the practitioner desires to maintain the cells in the washing buffer for an extended period, however, the osmolarity of the wash buffer should be relatively close to that of the loading solution to prevent the loaded solute from leaking from the cells into the wash buffer over time.

The amount of the preferred trehalose loaded inside the cells ranges from about 10 mM to about 50 mM, and is achieved by incubating the cells to preserve biological properties during drying. The effective loading of trehalose is also accomplished by using a temperature of from greater than about 25° C. to about 40° C., more preferably from about 30° C. to less than about 40° C., most preferably about 37° C.

After the cells have been effectively loaded with a solute and subsequently washed, the cells may then be contacted with a drying buffer. The drying buffer preferably includes the solute, preferably in amounts up to about 100 mM. The solute in the drying buffer assists in spatially separating the cells as well as stabilizing the cell membranes on the exterior. The drying buffer preferably also includes a bulking agent (to further separate the cells). The presence of albumin is desirable to serve as a spacer to keep the cells from contacting each other (spacing agents are sometimes referred to as “bulking agents”). Human serum albumin is preferred. Polymers may be used with or in place of albumin, although fewer cells will survive if albumin is absent. Suitable polymers, for example, are water-soluble polymers such as HES (hydroxy ethyl starch) and dextran.

The solute loaded cells in the drying buffer may then be dried while simultaneously cooled to a temperature below about −32° C. A cooling, that is, freezing, rate is preferably between −30° C. and −1° C./min. and more preferably between about −2° C./min to −5° C./min. Drying may be continued until about 95 weight percent of water has been removed from the cells. If the drying method selected is freeze-drying, during the initial stages of lyophilization, the pressure is preferably at about 10×10⁻⁶ torr. As the samples dry, the temperature can be raised to be warmer than −32° C. Based upon the bulk of the sample, the temperature and the pressure it can be empirically determined what the most efficient temperature values should be in order to maximize the evaporative water loss. Freeze-dried cell compositions preferably have less than about 5 weight percent water.

After drying the cells, the process of using such a dehydrated cell composition comprises rehydrating the cells. While we have not found much difference between prehydrating cells and simply rehydrating them, optionally, the rehydration can include a prehydration step, sufficient to bring the water content of the freeze-dried cells to between about 20 weight percent and about 50 percent, preferably from about 20 weight percent to about 40 weight percent. When prehydration is desired, the dried cells are prehydrated in moisture saturated air at about 37° C. for about one hour to about three hours, followed by rehydration.

For various embodiments of the present invention, the solute solution comprises: a solute and a salt solution. The concentration of the solute in the solute solution may be at least about 50 mM, such as ranging from about 50 mM to about 3000 mM, preferably from about 100 mM to about 1500 mM, more preferably from about 150 mM to about 1000 mM, most preferably from about 200 mM to about 600 mM. The osmolarity of the salt solution may be at least about 25 mOsm, such as ranging from about 25 mOsm to about 1 000 mOsm, preferably from about 50 mOsm to about 300 mOsm, more preferably from about 75 mOsm to about 200 mOsm. The solute solution comprising a solute and a salt solution may be used for any suitable purpose.

The salt solution may be any suitable physiologically acceptable solution in an amount and under conditions effective to function as a carrier medium for a solvent, or for a mixture of a solvent, a protein and/or an inert substance. The salt solution may comprise a phosphate buffered saline (PBS) solution comprising NaCl, Na₂BPO₄, and KH₂PO₄. A suitable PBS buffer is 100 mOsm PBS buffer (51.3 mM NaCl, 1.87 mM Na₂HPO₄, 0.35 mM KH₂PO₄, pH 7.2).

For additional various embodiments of the present invention, the solute solution may further comprise (in addition to the solute and the salt solution) a protein and/or an inert substance. The amount or quantity of the inert substance (e.g., HES) in the solute solution may be at least about 2.0% by weight, such as ranging from about 2.0% by weight to about 50% by weight, preferably from about 5% by weight to about 35% by weight, more preferably from about 10% by weight to about 30% by weight, most preferably from about 12% by weight to about 20% by weight (e.g., about 15% by weight). The amount or quantity of the protein (e.g. HSA) in the solute solution may be at least about 0.5% by weight, such as ranging from about 0.5% by weight to about 15% by weight, preferably from about 1% by weight to about 10% by weight, more preferably from about 1.5% by weight to about 8% by weight, most preferably from about 1.5% by weight to about 5% by weight (e.g., about 2.5% by weight). The solute solution comprising a solute, a salt solution, a protein and/or an inert substance may be used for any suitable purpose including as a freeze drying buffer and/or rehydration buffer.

The inert substance is preferably a carbohydrate, such as any of the carbohydrates previously mentioned above. Preferably, the inert substance comprises a polysaccharide. Preferably, the inert substance comprises a starch, such as, by way of example, hydroxy ethyl starch (HES).

The quantities of solute, protein and inert substance employed in the solute solution, more specifically in combination with a saline solution, are of suitable quantities and proportion for minimizing the loss or destruction of cells, more particularly for minimizing hemolysis, especially after drying and reconstitution (e.g., prehydration and rehydration), and/or especially when the solute solution is employed as a freeze-drying buffer and/or rehydration buffer.

When a solute is loaded from a solute solution into one or more cells, the solute solution preferably has a solute concentration of sufficient magnitude to produce hyperosmotic pressure on the one or more cells. It has been discovered that the basis for the loading of the solute into the cells is dependent upon osmotic shock. The magnitude of osmotic shock and hyperosmotic pressure on the cells depends on the difference between internal solute concentration, or the intracellular osmolarity, within the cells, and the external solute concentration within the solute solution, or the extracellular cellular solute concentration. For embodiments of the invention, the solute solution has a solute concentration ranging from about 500 mM to about 1500 mM, preferably from about 600 mM to about 1300 mM, more preferably from about 700 mM to about 1000 mM.

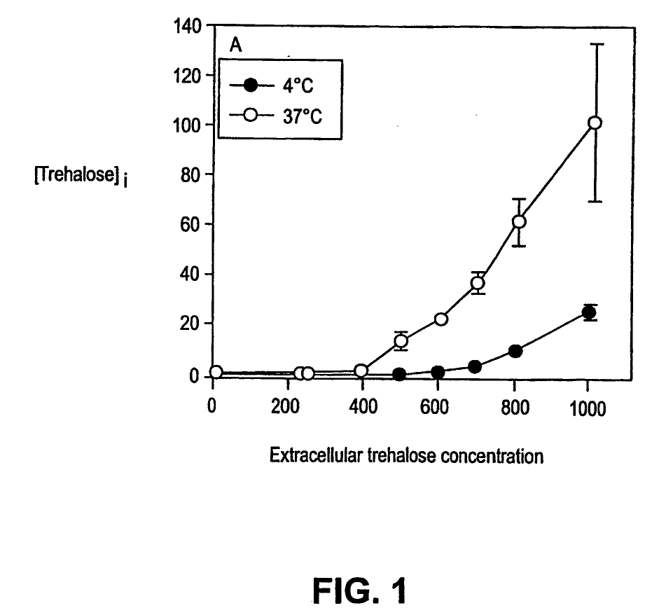

It has also been discovered that the basis for the loading of the solute into the cells is not only dependent upon osmotic shock, but is also dependent upon the thermal effects on flux of the solute across the membranes of the cells. The higher the thermal effects on flux of the solute across the membranes of the cells, the larger the amount of solute loaded into the cells. Stated alternatively, up to a point, loading of a solute into cells increases as the temperature of the solute solution increases. Referring now to FIG. 1, there is seen a graphical illustration of intracellular trehalose concentration as a function of extracellular trehalose concentration at respective temperatures of 4° C. and 37° C. Thus, at a temperature ranging from about 30° C. to about 40° C. (e.g. at about 37° C.) a gradient of a solute concentration (mM), such as an oligosaccharide (e.g., trehalose) concentration, within a cell (e.g., an erythrocytic cell) to extracellular solute concentration (mM) within a loading solution (or buffer) ranges from about 0.130 to about 0.200. At a temperature ranging from about 0° C. to about 10° C. (e.g. at about 4° C.) a gradient of a solute concentration (mM), such as an oligosaccharide (e.g., trehalose) concentration, within a cell to extracellular solute concentration (mM) within a loading solution (or buffer) ranges from about 0.04 to about 0.12, more specifically from about 0.04 to about 0.08, and from about 0.08 to about 0.12, depending on the quantity of extracellular solute concentration. Example 1 below provides the more specific testing conditions and parameters which produced the graphical illustrations of FIG. 1.

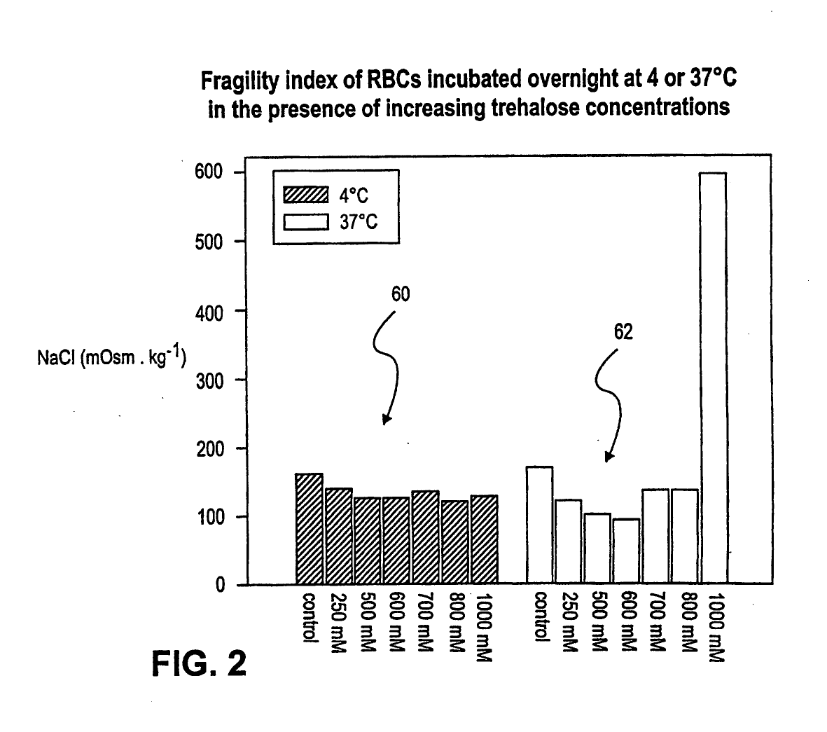

Referring now to FIG. 2, there is seen a graphical illustration of the fragility index of erythrocytic cells incubated overnight at respective temperatures of 4° C. and 37° C. in the presence of and as a function of increasing extracellular trehalose concentrations. The osmotic fragility index was generated by the extent of hemolysis as a function of the NaCl concentration. The graphical illustration of FIG. 2 represents a test for investigating the effects of hyperosmotic treatment rendering erythrocytic cells more sensitive to change in intracellular osmolarity. NaCl was loaded into erythrocytic cells from a 100 mOsm PBS buffer at loading 100 mOsm PBS buffer temperatures of 4° C. and 37° C. for extracellular trehalose concentrations of 0 mM (control cells), 250 mM, 500 mM, 600 mM, 700 mM, 800 mM and 1000 mM. Data blocks, respectively generally indicated as 60 and 62, represent the intracellular trehalose concentrations for 100 mOsm PBS solution loading temperatures of 4° C. and 37° C. The mOsm/kg values of NaCl represent extracellular NaCl osmolarity of the erythrocytic cells resulting from the transfer of NaCl from the PBS loading buffer into the erythrocytic cells. The erythrocytic cells that had been loaded in trehalose solutions (between 250 mM and 1000 mM) in 100 mOsm PBS were suspended in increasing concentrations of NaCl (between 50 and 600 mOsm NaCl). The percent hemolysis measured after resuspending the loaded cells in NaCl represents the fragility index. The data show that the erythrocytic cells were stable osmotically in trehalose media with concentrations between 250 mM and 800 mM trehalose at both 37° C. and 4° C. In 1000 mM trehalose at 37° C., there is a high increase in the fragility index suggesting that the cells were unstable in this medium (1000 mM trehalose in 100 mOsm PBS). Clearly, at moderate intracellular concentrations of trehalose, osmotic fragility as measured by a standard assay was not severely altered. Thus, erythrocytic cells may be loaded with trehalose concentrations up to about 900 mM (i.e., a trehalose concentration between 800 mM and 1000 mM). Example 2 below provides specific testing conditions and parameters which produced the graphical illustrations of FIG. 2.

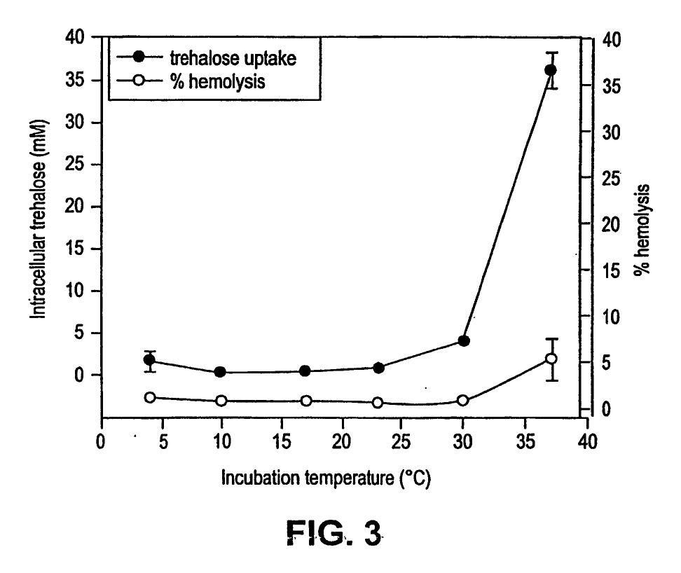

Thus, from the findings graphically illustrated in FIGS. 1 and 2, and as more fully explained in Examples 1 and 2 below, temperature of a solute loading solution has an effect in loading a solute from a solute solution into a cell. The effects of temperature, as well as cellular hemolysis, of a trehalose loading solution in loading of trehalose into a cell was tested. The test results are illustrated in FIG. 3, which is a graphical illustration of trehalose uptake (i.e., intracellular trehalose mM) and hemolysis (i.e., % hemolysis) as a function of incubation temperature (° C.). FIG. 3 illustrates that effective loading occurs above 30° C., and that as the loading temperature of the trehalose loading solution increases, there is slight hemolysis. Example 3 below provides the more specific testing conditions and parameters which produced the graphical illustrations of FIG. 3.

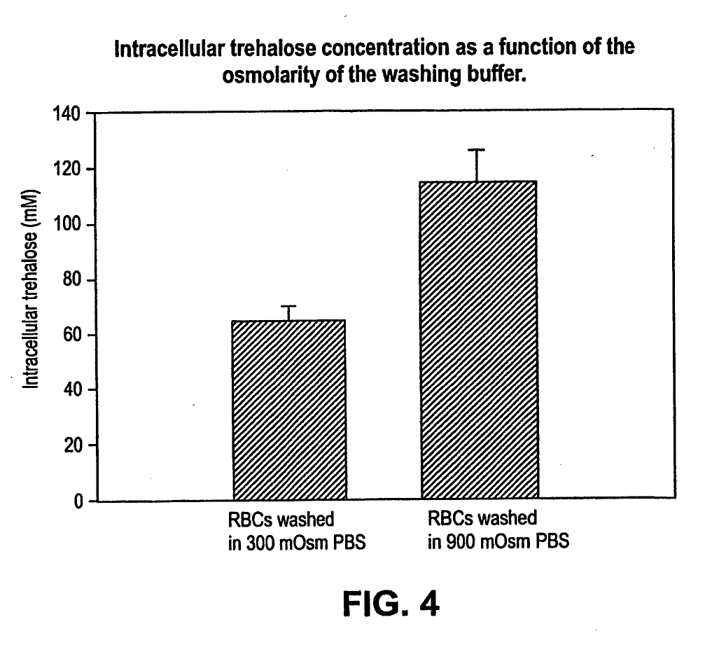

As previously indicated, after a cell (e.g., an erythrocytic cell) has been loaded with a solute (e.g., trehalose), further embodiments of the present invention provide for retaining the solute in the cells. One means for retaining solute within solute-loaded cells is to wash the cells, more specifically by washing the cells and retaining the solute in the cells during the washing. As also previously indicated, the washing of the cells is preferably with a washing buffer. It has been discovered that retention of the solute in the cells increases from about 25% to about 175% when a buffer concentration (e.g., the osmolarity of all osmotically active particles within the washing buffer solution) increases from about 50% to about 400%, more preferably from about 50% to about 150% when a buffer concentration increases from about 100% to about 300%, and most preferably from about 75% to about 125% (e.g., about 100%) when a buffer concentration increases from about 150% to about 250% (e.g., about 200%). It has been further discovered that the washing of the cells with a washing buffer includes employing a ratio of an extracellular buffer concentration (mOsm) to an intracellular trehalose concentration (mM) ranging from about 14.0 to about 4.0, more particularly from about 12.0 to about 5.0, including from about 9.0 to about 6.0 and from about 8.0 to about 7.0 (e.g., about 7.5). Thus, because solute loaded cells are hyperosmotic to a washing buffer, increasing the extracellular osmolarity increases retention of the solute during washing of the cells, as shown in FIG. 4 which graphically illustrates trehalose uptake (i.e., intracellular trehalose mM) as a function of the osmolarity of the washing buffer. As shown in FIG. 4, when the extracellular buffer concentration was increased from 300 mOsm PBS to 900 mOsm PBS during washing, the final intracellular trehalose concentration doubled. The cells that were washed with 300 mOsm PBS had a 65 mM trehalose concentration, where as the cells that were washed with 900 mOsm PBS had a 115 mM intracellular trehalose concentration. Example 4 below provides the more specific testing conditions and parameters which produced the graphical illustrations of FIG. 4.

After the cells have been effectively loaded with a solute and subsequently washed, the cells may then be contacted with a drying buffer. The drying buffer preferably includes the solute, preferably in amounts up to about 100 mM. The solute in the drying buffer assists in spatially separating the cells as well as stabilizing the cell membranes on the exterior. The drying buffer preferably also includes a bulking agent to further assist in separating the cells. Albumin may serve as a bulking agent, but other polymers may be used with the same effect. If albumin is used, it may be from the same species as the cells. Suitable other polymers, for example, are water-soluble polymers such as HES (hydroxy ethyl starch) and dextran.

The solute-loaded cells in the drying buffer may then be dried. Preferably, the solute loaded cells are dried while simultaneously cooling to a temperature below about −32° C. A cooling, that is, freezing, rate is preferably between −30° C. and −1° C./min. and more preferably between about −2° C./min to −5° C./min. Drying may be continued until about a residual water content of about 3% is achieved. During the initial stages of lyophilization, the pressure is preferably at about 10×10⁻⁶ torr. As the samples dry, the temperature can be raised to be warmer than −32° C. Based upon the bulk of the sample, the temperature and the pressure it can be empirically determined what the most efficient temperature values should be in order to maximize the evaporative water loss. Dried cell compositions preferably have less than about 5 weight percent water.

After drying and storage of the cells, the process of using such a dehydrated cell composition comprises rehydrating the cells. Rehydration of the prehydrated cells may be with any aqueous based solutions, depending upon the intended application.

Intracellular trehalose, more particularly intracellular trehalose along with intracellular inert substance and protein, improve the survival of rehydrated cells. The concentration of intracellular trehalose in rehydrated cells is also important for subsequent stabilization of the rehydrated cells. As the concentration of intracellular trehalose increases for rehydrated erythrocytic cells, the mean corpuscular hemoglobin (MCH, the amount of hemoglobin found in intact erythrocytic cells) also increases. An intracellular trehalose concentration (mM) of about 40 mM and (or to) about 42 mM produces an MCH of about 9 (pg). When the intracellular trehalose concentration (mM) increases to about 60 mM, the MCH precipitously increases to above about 10 (pg), more specifically above or greater than about 14 (pg). Thus, embodiments of the present invention include loading cells with an effective amount of solute for stabilizing cells. An effective amount of a solute is greater than about 50 mM, such as 60 mM or above. Furthermore, as intracellular concentration of trehalose increases, there is a significant decrease in the percent (%) hemolysis, to an extent of less than about 10%, and even less than about 5%. As cytoplasmic intracellular trehalose approaches a concentration of about 100 mM, % hemolysis falls to below about 10%.

The following protocol has been discovered as yielding significant survival of freeze-dried cells. The loading buffer comprised about 800 mM trehalose in a salt solution of about 100 mOsm PBS. The incubation time was about 16 hours at a temperature of about 35° C. After the cells were loaded, they were subsequently washed in a washing buffer comprising about 300 mM trehalose in a salt solution of about 100 mOsm PBS. Within about 3 hours after washing the loaded cells, the wash loaded cells were freeze-dried in freeze-drying buffer comprising about 300 mM trehalose, about 100 mOsm PBS, about 2.5% by wt. HSA, and about 15% by wt. HES. After freeze-drying, the cells had about 75 mM trehalose, about 25 mOsm PBS, about 0.6% by wt. HSA and about 4.0% by wt. HES left in the cells. In various embodiments of the invention for producing maximal survival of the cells, the dried cells comprise from about 25 mM to about 300 mM trehalose, from about 5 mOsm to about 100 mOsm osmolarity for the salt solution, from about 0.1% by weight to about 2.5% by weight of the protein, and from about 1.0% by weight to about 15.0% by weight of the inert substance; and preferably from about 60 mM to about 80 mM trehalose, from about 10 mOsm to about 40 mOsm PBS, from about 0.3% by weight to about 9.0% by weight albumin, and about 1.0% by weight to about 4.0% by weight starch. The freeze-dried cells were then reconstituted at about 37° C. for about 10 minutes in a rehydration buffer comprising about 188 mM trehalose, about 100 mOsm PBS, about 2.5% by wt. HSA and about 15.0% by wt. HES. After rehydration, less than about 5% of the cells were lysed.

Embodiments of the present invention will be illustrated by the following set forth examples which are being given to set forth the presently known best mode and by way of illustration only and not by way of any limitation. It is to be understood that all materials, chemical compositions and procedures referred to below, but not explained, are well documented in published literature and known to those artisans possessing skill in the art. All materials and chemical compositions whose source(s) are not stated below are readily available from commercial suppliers, who are also known to those artisans possessing skill in the art. All parameters such as concentrations, mixing proportions, temperatures, rates, compounds, etc., submitted in these examples are not to be construed to unduly limit the scope of the invention. Abbreviations used in the examples, and elsewhere, are as follows:

DMSO=dimethylsulfoxide

ADP=adenosine diphosphate

PGE1=prostaglandin E1

HES=hydroxy ethyl starch

FTIR=Fourier transform infrared spectroscopy

EGTA=ethylene glycol-bis(2-aminoethyl ether) N,N,N′,N′, tetra-acetic acid

TES=N-tris(hydroxymethyl)methyl-2-aminoethane-sulfonic acid

HEPES=N-(2-hydroxyl ethyl)piperarine-N′-(2-ethanesulfonic acid)

PBS=phosphate buffered saline

HSA=human serum albumin

BSA=bovine serum albumin

ACD=citric acid, citrate, and dextrose

MβCD=methyl-β-cyclodextrin

### EXAMPLE 1

FIG. 1 graphically illustrates the loading efficiency of trehalose into human erythrocytic cells as a function of external trehalose concentration at respective temperatures of 4° C and 37° C. Erythrocytic cells were exposed to trehalose for 18 hours at either 4° C. or 37° C. The trehalose concentration in the incubation medium varied between 230 mM and 1000 mM. Each incubation buffer contained trehalose (between 230 mM nd 1000 mM) and 100 mOsm PBS pH 7.2. Increase in the trehalose concentration in the loading medium results in an increase in the sugar uptake, reaching about 100 mM cytoplasmic trehalose in erythrocytes incubated in 1000 mM trehalose and 100 mOsm PBS. At 4° C., the uptake was very limited, being about 25 mM. The trehalose intake was measured using anthrone assay and confirmed by high performance liquid chromatography. It is clear that there was substantial loading at 37° C., but not at 4° C. Furthermore, trehalose loading was not significant unless the extracellular cellular trehalose concentration gave a hyperosmotic pressure. Since intracellular osmolarity for erythrocytic cells is about 300 mOsm, it is clear that raising the extracellular osmolarity was required for more effective loading of trehalose.

### EXAMPLE 2

FIG. 2 graphically illustrates the fragility index of erythrocytic cells incubated overnight at respective temperatures of 4° C. and 37° C. in the presence of and as a function of increasing intracellular trehalose concentrations. The osmotic fragility index was generated by the extent of hemolysis as a function of the NaCl concentration. The erythrocytic cells that had been loaded in trehalose solutions (between 250 mM and 1000 mM) in 100 mOsm PBS were suspended in increasing concentrations of NaCl (between 50 and 600 mOsm NaCl). The percent hemolysis measured after resuspending the loaded cells in NaCl represents the fragility index. The data show that the erythrocytic cells were stable osmotically in trehalose media with concentrations between 250 mM and 800 mM trehalose at both 37° C. and 4° C. In 1000 mM trehalose at 37° C., there is a high increase in the fragility index suggesting that the cells were unstable in this medium (100 mM trehalose in 100 mOsm PBS).

### EXAMPLE 3

FIG. 3 graphically illustrates trehalose uptake (i.e., intracellular trehalose mM) and hemolysis (i.e., % hemolysis) as a function of incubation temperature (° C.). The incubation temperature was varied between 4° C. and 37° C. The erythrocytic cells were incubated for 6 hours in 800 mM trehalose in 100 mOsm PBS pH 7.2. Between 4° C. and 30° C., the cytoplasmic trehalose was very low (between 1 and 4 mM). It was considerably increased (up to 35 mM cytoplasmic trehalose) during 6 hours incubation at 37° C.

### EXAMPLE 4

FIG. 4 graphically illustrates intracellular trehalose (mM) as a function of the osmolarity of the washing buffer. Earlier morphological data showed that along with biconcave discoid erythrocytic cells, about 20% of cells have a modified shape (spherocytes and schistocytes). The issue was what was the loading capacity of these cells and how much they contribute to the amount of trehalose that was to be detected. This issue was investigated by washing the trehalose loaded erythrocytic cells (loaded at 35° C. for 16 hours in 800 mM trehalose in 100 mOsm PBS pH 7.2) in buffers with different osmolarity (300 mOsm PBS or 900 mOsm PBS) and estimating the cytoplasmic sugar concentration. The loaded cells were washed with either 300 mOsm PBS pH 7.2 (which is the isotonic medium for erythrocytic cells) or 900 mOsm PBS pH 7.2 (which matches the tonicity of the loading medium). The data in FIG. 4 illustrates that there was a decrease in the intracellular sugar concentration, suggesting that a fraction of the cells was lost during the washing procedure.

### EXAMPLE 5

The following loading protocol has been discovered as yielding significant survival of freeze-dried cells. The loading protocol includes incubating the erythrocytic cells in 800 mM trehalose, 100 mOsm ADSOL® and 6.6 mM Na-phosphate. ADSOL® comprises 111 mM glucose, 2 mM adenine, 154 mM NaCl and 41 mM mannitol. The incubation temperature for loading was between 38 and 41° C., and the time of incubation was 6 hours. This loading procedure yielded lower extent of hemolysis (about 17%), as compared to the hemolysis measured during loading in 800 mM trehalose and 100 mOsm PBS for 16 hours at 37° C. Furthermore, this loading procedure was not accompanied by significant changes in cell morphology. At the same time, the amount of intracellular trehalose was the same as during loading erythrocytes in 800 mM trehalose and 100 mOsm PBS at 37° C. for 16 hours. No washing was applied after termination of the loading step and prior to freeze-drying. Immediately after completing the loading, the cells were mixed gently with the freeze-drying buffer. The final concentration of the freeze-drying buffer was 250 mM trehalose, 20 mOsm ADSOL®, 15% HES and 2.5% human serum albumin (HSA). The freeze-dried cells were rehydrated at 37° C. for about 10 rnin in a rehydration buffer containing 141 mM trehalose, 75 mOsm PBS 11.25% HES and 1.875% HSA.

During the loading step, the levels of the following two important metabolites were followed: adenosine-3-phosphate (ATP) and 2,3-diphosphoglycerate (2,3-DPG). ATP level correlates with the efficiency of the glycolic pathway which is the major biochemical pathway in erythrocytes. The polyanion 2,3-DPG binds to the central cavity of the hemoglobin tetramer and modulates the affinity of hemoglobin for oxygen. It is important for the oxygen carrying capacity of hemoglobin. The normal level of ATP in freshly isolated erythrocytes was between 3.65 and 4.45 μmole/g Hb. The ATP level of erythrocytes in buffers with different compositions was followed during 5 hours incubation at 38-41° C. During incubation in 100 mOsm ADSOL® and 6.6 mM Na-phosphate or in 800 mM trehalose, 100 mOsm ADSOL® and 6.6 mM Na-phosphate, the measured ATP level was very similar to that of freshly isolated erythrocytes. When erythrocytes were incubated in 800 mM trehalose and 100 mOsm PBS, the level of ATP was also as high as in fresh cells. It was slightly reduced when cells were incubated in 800 mM trehalose and 100 mOsm ADSOL® (without Na-phosphate), and when the cells were incubated in ADSOL® only (462 mOsm).

### EXAMPLE 6

The level of 2,3-DPG was followed during 5 hours incubation at 38-41° C. in buffers with different composition. The normal level of 2,3-DPG in freshly isolated erythrocytes is around 12.8 μmole/g Hb. The highest 2,3-DPG level was observed in cells incubated in 800 mM trehalose, 100 mOsm ADSOL® and 6.6 mM Na-phosphate and in 800 mM trehalose and 100 mOsm ADSOL®. It was decreased for cells incubated in ADSOL® (462 mOsm), in ADSOL® and 6.6 mM Na-phosphate, in 800 mM trehalose and 100 mOsm PBS and in 300 mOsm PBS.

On the basis of the observations, an incubation medium comprising 800 mM trehalose, 100 mOsm ADSOL® and 6.6 mM Na-phosphate provides high levels of ATP and 2,3-DPG.

Pre-hydration via exposure to water vapor produces a gradual and more homogenous rehydration of dried biomaterials than direct rehydration. Erythrocytic cells were loaded in 800 mM trehalose, 100 mOsm ADSOL® and 6.6 mM Na-phosphate at 38-41° C. for 6 hours and were freeze-dried in a buffer with a final concentration of 250 mM trehalose, 20 mOsm ADSOL®, 15% HES and 2.5% HSA. Freeze-dried cells were pre-hydrated for various times (between 5 and 30 mins.) and then they were rehydrated at 37° C. for 10 min in a buffer containing 141 mM trehalose, 75 mOsm PBS, 11.25% HES and 1.875% HSA. Pre-hydration for 5 min at 37° C. resulted in a lower percent of hemolysis.

### EXAMPLE 7

α-crystallin is a member of the small heat shock protein family and is highly abundant in a number of mammalian cell types and tissues. It has been discovered that α-crystallin associates with lipid membranes in vitro and preserves their integrity at high non-lethal temperatures. The effect of a-crystallin on the percent hemolysis was studied. Cells were loaded in either 800 mM trehalose, 100 mOsm ADSOL®, 6.6 mM Na-phosphate, or in 800 mM trehalose, 100 mOsm ADSOL®, 6.6 mM Na-phosphate and 1.2 mg/ml α-crystallin. Cells were subsequently mixed with freeze-drying buffer with final concentration of 250 mM trehalose, 20 mOsm ADSOL®, 15% HES and 2.5% HSA and were freeze-dried. After freeze-drying they were directly rehydrated (no pre-hydration) in 141 mM trehalose, 75 mOsm PBS, 11.25% HES and 1.875% HSA. Cells loaded in the presence of 1.2 mg/ml α-crystallin show lower percent hemolysis (49%) in comparison to those loaded without α-crystallin (68%). In a further study, along with 1.2 mg/ml α-crystalhn, 0.5 mg/ml α-crystallin was added to the rehydration buffer. The data show that the addition of α-crystallin to the rehydration buffer does not result in higher cell survival after rehydration. The conclusion from these data is that α-crystallin in the loading buffer improves the survival of freeze-dried and rehydrated erythrocytic cells, as assessed by the decrease in hemolysis from 68% (in cells that have not been loaded in the presence of α-crystallin) to 49% (in cells loaded in the presence of α-crystallin).

### EXAMPLE 8

It has been further discovered that transition metal ions decrease hemolysis. When divalent ions Zn²⁺ ions are added to the rehydration buffer, there is a decrease in the percent hemolysis of rehydrated erythrocytic cells. Zn²⁺ ions stabilize thermally labile enzymes during drying. Rehydration experiments were performed combining α-crystallin and Zn²⁺ ions, and applying 5 min pre-hydration. Under these conditions, 62% of the cells survived the rehydration step, indicating that the beneficial effect of these treatments is additive. When the cells were loaded in 800 mM trehalose, 100 mOsm ADSOL and 6.6 mM Na-phosphate, data set labeled as T), freeze-dried in 250 mM trehalose, 20 mOsm ADSOL, 15% HES and 2.5% human serum albumin (HAS) and directly rehydrated at 37° C. for 10 min in 141 mM trehalose, 75 mOsm PBS, 11.25% HES and 1.875% HSA, the hemolysis was 63%. When the cells were loaded in a buffer containing 800 mM trehalose, 100 mOsm ADSOL, 6.6 mM Na-phosphate and α-crystallin (1.2 mg/ml), freeze-dried in 250 mM trehalose, 20 mOsm ADSOL, 15% HES and 2.5% HSA, pre-hydrated at 37° C. for 5 min, and then fully rehydrated at 37° C. for 10 min in 141 mM trehalose, 75 mOsm PBS, 11.25% HES and 1.875% HSA, the hemolysis was 47%. When cells were loaded in a buffer containing 1.2 mg/ml α-crystallin, 800 mM trehalose, 100 mOsm ADSOL, 6.6 mM Na-phosphate, freeze-dried in 250 mM trehalose, 20 mOsm ADSOL, 15% HES and 2.5% HSA, pre-hydrated for 5 min at 37° C., and rehydrated at 37° C. for 10 min in a buffer containing 500 μM ZnSO₄, 141 mM trehalose, 75 mOsm PBS, 11.25% HES and 1.875% HSA, the hemolysis was only 38%, giving rise to 62% cell survival.

### EXAMPLE 9

The levels of ATP and 2,3-DPG were followed during rehydration of freeze-dried erythrocytic cells. Incubation of the rehydrated cells in a buffer supplemented with rejuvenation solution led to considerable increase in the ATP and 2,3-DPG synthesis. The levels of the two metabolites were followed during 10 min and 60 min incubations at 37° C. in a rehydration buffer containing 141 mM trehalose, 15% HES, 2.5% HSA and the following rejuvenation supplements: 100 mM pyruvate, 100 mM inosine, 100 mM Na-phosphate and 5 mM adenine. The rejuvenation solution is referred as “PIPA”. Cells were loaded in 800 mM trehalose, 100 mOsm ADSOL and 6.6 mM Na-phosphate at 38-41° C. for 6 hours. They were freeze-dried in 250 mM trehalose, 20 mOsm ADSOL, 15% HES and 2.5% HSA, and rehydrated at 37° C. for 10 min in 141 mM trehalose, 75 mOsm PBS, 11.25% HES and 1.875% HSA. Without applying rejuvenation solution, the levels of both ATP and 2,3-DPG are low. However, when such cells were rehydrated in buffer containing 141 mM trehalose, 75 mOsm PBS, 11.25% HES and 1.875% HSA, supplemented with PIPA, the levels of ATP and 2,3-DPG were increased. These results show that supplementation of the rehydration medium with 100 mM pyruvate, 100 mM inosine, 100 mM Na-phosphate and 5 mM adenine increases the synthesis of these two vital metabolites and can be applied during reconstitution 7of freeze-dried erythrocytic cells.

### EXAMPLE 10

Freshly isolated red blood cells (RBCs) contain less than 1% methemoglobin (metHb), although its percentage may vary depending on the donor. Trehalose-loaded RBCs contain about 7% metHb. The data show that ascorbic acid reduces considerably the percentage of metHb.

RBCs were loaded in a loading buffer composed of 800 mM trehalose, 100 mOsm ADSOL and 6.6 mM K-phosphate and contained 1, 5 or 8 mM ascorbic acid. RBCs were incubated at 37° C. for 7 hours. The control cells were isolated and stored in ADSOL (460 mOsm).

Since ascorbic acid is a strong reducing agent, it induces a change in the pH of the extracellular medium. We followed the pH of the extracellular medium as a function of the ascorbic acid during resuspension of the RBCs in loading, freeze-drying and rehydration buffers. In these experiments, loading, freeze-drying and rehydrating media were buffered with 6.6 mM K-phosphate (pH 7.2). The data show a rapid decrease of the extracellular pH in the presence of increasing concentration of ascorbic acid. However, between 0.125 mM and 5 mM ascorbic acid, the pH is maintained close to 6.5, therefore we used 5 mM ascorbic acid in the rehydration medium.

In order to determine an optimum concentration of ascorbic acid that produces a decrease in the percent metHb, we studied the percent metHb as a function of the concentration of ascorbic acid. RBCs were loaded in 800 mM trehalose, 100 mOsm ADSOL and 6.6 mM K-phosphate, and freeze-dried in 15% HES, 2.5% HSA, 300 mM trehalose and 6.6 mM K-phosphate. The rehydration buffer contained 141 mM trehalose, 11.25% HES, 1.875% HSA, 75 mOsm PBS, 6.6 mM K-phosphate and the test amount of ascorbic acid. In some experiments, 30 mM K-phosphate was used in the rehydration buffer, instead of 6.6 mM K-phosphate.

In the presence of 6.6 mM K-phosphate and between 1 and 6 mM ascorbic acid, metHb decreased from 35% to 20%. Further increase in the concentration of ascorbic acid between 8 and 18 mM resulted in an increase in the percent metHb, showing that ascorbic acid has a dual effect. Based on our previous studies, we suggest that this effect is associated with a decrease in pH, which is detrimental for the cell viability. On the basis of these results, it appears 5 mM ascorbic acid in the rehydration medium is desirable.

Further, we studied the effect of ascorbic acid on the percent metHb in freeze-dried RBCs. Ascorbic acid reduces considerably the percent of metHb in rehydrated RBCs (from about 50% to 15% methb in trehalose loaded RBCs), especially when it is present in the rehydration buffer. Further, the color and morphology of the rehydrated cells is considerably improved when the rehydration medium is supplemented with 2 mM ascorbic acid.

### EXAMPLE 11

Methylene blue is a drug used clinically for reducing the level of methemoglobin. The mechanism of action of the drug includes a reduction of the dye to leukomethylene blue by means of NADH (formed in the hexose monophosphate pathway). Leukomethylene blue, in turn, reduces methemoglobin nonenzymatically to hemoglobin. FIG. 6 presents the percent hemolysis and percent metHb in freeze-dried trehalose loaded RBCs in the presence and absence of methylene blue. There was a reduction in the percent of metHb from 18% to 9% when 10 mM methylene blue was added to the rehydration medium (FIG. 6).

FIG. 6. Effect of methylene blue on the percent methemoglobin and percent hemolysis in freeze-dried trehalose loaded RBCs. The rehydration buffer contained 141 mM trehalose, 75 mM PBS, 11.3% HES, 1.88% HSA, 5 mM ascorbic acid, and was supplemented with 10 μM methylene blue (MB). The rehydration time was 10 min and the temperature for rehydration was 37° C.

| Sample | | | | |
|---|---|---|---|---|
| treatment | % hemolysis | SD | % hemolysis | SD |
| (−MB) | 52.2 | 0.56 | 18.2 | 3.35 |
| (+MB) | 50.7 | 1.04 | 8.7 | 4.62 |

### EXAMPLE 12

This Example shows the effect of cell concentration (hematocrit) on the survival of freeze-dried RBCs.

We studied the effect of the concentration of RBCs during freeze-drying on their survival after rehydration. The data show that decreased cell concentration during freeze-drying resulted in lower percent hemolysis, and therefore higher cell survival. In all cases, the mass ratio between trehalose, HES and HSA was 1:1.3:0.5, respectively.

The effect of cell concentration (hematocrit) of trehalose loaded RBCs was tested during freeze-drying. In this experiment, increasing concentrations of trehalose loaded RBCs were resuspended in a freeze-drying buffer containing 250 mM trehalose, 20 mOsm ADSOL, 15% HBS and 2.5% HSA. They were freeze-dried and rehydrated according to our standard procedure.

The results are in agreement with earlier studies on the effect of cell concentration on the survival of RBCs during freezing and thawing. One possible reason for the observed effect may be the cell packing during freezing and drying. Without wishing to be bound by theory, it seems likely that cell-cell contacts induced by freezing lead to membrane interactions that may be damaging, and that these interactions are responsible for packing injury. At the same time, the metabolic activity of the rehydrated RBCs assessed by measuring the level of 2,3-DPG and ATP remains very similar for the two cell concentrations (5% and 15%), suggesting that the damage is most likely physical rather than physiological. Although multiple factors such as cell packing or/and solute availability may possibly contribute to the packing injury during freeze-drying, cell membrane interactions may well be among the most harmful.

We further investigated the percent hemolysis of freeze-dried RBCs as a function of the concentration of trehalose in the freeze-drying buffer. In this experiment, we compared hematocrits of about 2.5%, 5%, 7.5% and 15% during freeze-drying. The mass ratio between HES and albumin remained constant. The data showed that 100 mM trehalose in the freeze-drying buffer resulted in lowest percent hemolysis, thus, highest cell survival. In all cases, the percent hemolysis using 5% hematocrit was lower that 15% hematocrit. At the same time, the percent metHb remained unchanged, irrespective of the concentration of trehalose in the freeze-drying buffer or the percent hematocrit. HES and albumin serve as excipients in the freeze-drying buffer and help prevent aggregation of RBCs during freeze-drying and rehydration. They increase the Tg in combination with trehalose. Reducing the extracellular trehalose in the freeze-drying buffer may lead to reducing the osmotic pressure caused by the disaccharide. In addition, it is balancing the concentration of the sugar between the cytoplasm and the extracellular milieu, resulting in higher survival after lyophilization.

On the basis of these results, a hematocrit of 2-5% hematocrit is desirable during freeze-drying of trehalose loaded RBCs. The freeze-drying buffer contains 100 mM trehalose, 100 mOsm ADSOL, 15% HES, 2.5% HSA and 6.6 mM K-phosphate.

### EXAMPLE 13

This Example demonstrates the effect of substituting trehalose with K-phosphate or KCl during freeze-drying and rehydration increases the survival of RBCs.

Trehalose loaded RBCs, washed with ADSOL (462 mOsm), were resuspended in freeze-drying buffer that contained increasing concentrations of K-phosphate or KCl, along with 15% HES, 2.5% HSA and 100 mOsm ADSOL. The data show that when 15% hematocrit is used during the freeze-drying step, the percent of RBC hemolysis after rehydration is reduced with 20% in the presence of K-phosphate, and with 5% in the presence of KCl, as compared to 5% hematocrit. This finding allows the use of a 15% hematocrit and obtaining a high percent survival after freeze-drying.

### EXAMPLE 14

This Example shows the effect of lipid vesicles (liposomes) on the survival of RBCs during freeze-drying and rehydration.

In this study, we supplemented the freeze-drying medium with liposomes composed of different phospholipid species. We investigated the percent hemolysis as a function of the concentration of lipids in the freeze-drying buffer.

Liposomes were prepared by sonication the lipids (20 mg/ml) in a buffer containing 800 mM trehalose, 100 mOsm Adsol and 6.6 mM K-phosphate. Trehalose-loaded RBCs were incubated for 10 min at room temperature with the liposomes before suspending them in freeze-drying buffer. The data show a strong decrease in the percent hemolysis of RBCs, therefore increased survival. The pattern is independent of the type of lipids (PC, PC/PS and PC/Cholesterol) that was used during the procedure.

The effect of different saturated phosphatidylcholines on the percent hemolysis of RBCs after freeze-drying and rehydration was investigated. Trehalose loaded RBCs were incubated with different species of saturated lipids (2 mg/ml) for 10 min prior to mixing with the freeze-drying buffer. The lipid concentration in the freeze-drying buffer was 0.245 mg/ml.

The data show that liposomes prepared from saturated PCs did not confer the same level of protection as unsaturated PC liposomes. DLPC, DMPC and DPPC resulted in approximately 50% hemolysis after freeze-drying and rehydration of RBCs, similar to control RBCs (un-treated with liposomes).

The effect of liposomes on the formation of methemoglobin during rehydration of the freeze-dried RBCs was also determined. The data show that liposomes do not affect significantly the level of methemoglobin during freeze-drying and rehydration.

Long-term stability of RBCs that were freeze-dried in the presence of egg PC liposomes was investigated. RBCs were stored at 4° C. up to 4 weeks and were rehydrated using standard conditions (described above). Lipid concentration in the freeze-drying buffer was 0.245 mg/ml. Trehalose loaded RBCs that were freeze-dried in the presence of egg PC liposomes (0.245 mg/ml) maintained their initial survival for up to four weeks at 4° C. At the same time, RBCs that were not loaded and were freeze-dried with and without egg PC liposomes, followed a trend similar to that of trehalose loaded RBCs, with a slightly higher extent in hemolysis. There was no increase in the percent methemoglobin in RBCs that were loaded with trehalose and freeze-dried in the presence of liposomes.

Trehalose loaded RBCs, freeze-dried with egg PC liposomes showed an increased level of survival (75±5%) after rehydration. The cells were viable for up to four weeks. No additional methemoglobin was formed during the storage at 4° C. for up to four weeks.

### EXAMPLE 15

This Example shows the effect of reducing the concentration of HES in the rehydration buffer and maintaining high viability of the rehydrated RBCs.

HES is a plasma substitute that is applied in transfusion medicine at a maximum concentration of 6%. It would be desirable to reduce the concentration of HES in rehydration buffer while maintaining high levels of viability in the freeze-dried RBCs. To this end, we performed a series of experiments in which we diluted the HES, by using isotonic PBS. The data showed that by reducing the amount of HES to 7.5%, the percent hemolysis was close to that observed in rehydration buffer. RBCs were loaded with trehalose in a buffer containing 800 mM trehalose, 100 mOsm ADSOl and 6.6 mM K-phosphate. They were freeze-dried in 15% HES, 2.5% HSA, 6.6 mM K-phosphate, 100 mOsm ADSOL, 100 mM trehalose, using 5% hematocrit. The rehydration buffer contained 11.25% HES, 1.875% HSA, 75 mOsm PBS, 141 mM trehalose, 6.6 mM K-phosphate and 5 mM ascorbic acid. Dilution was done using 50% Dulbecco PBS (300 mOsm) and 50% rehydration buffer.

While the present invention has been described herein with reference to particular embodiments thereof, a latitude of modification, various changes and substitutions are intended in the foregoing disclosure, and it will be appreciated that in some instances some features of the invention will be employed without a corresponding use of other features without departing from the scope and spirit of the invention as set forth. Therefore, many modifications may be made to adapt a particular situation or material to the teachings of the invention without departing from the essential scope and spirit of the present invention. It is intended that the invention not be limited to the particular embodiment disclosed as the best mode contemplated for carrying out this invention, but that the invention will include all embodiments and equivalents falling within the scope of the appended claims.

All publications, patents, and patent applications cited herein are hereby incorporated by reference in their entirety for all purposes.

What is claimed is:

1. A method for loading a solute into an erythrocytic cell, comprising:
disposing an erythrocytic cell in a solution having a solute concentration of sufficient magnitude to produce hyperosmotic pressure on the cell, thereby transferring a solute from the solution into the cell.

2. The method of claim 1 wherein said solute is present in said solution in a concentration of between 700 and 1000 mM.

3. The method of claim 1, wherein said solute is a disaccharide.

4. The method of claim 3, wherein said disaccharide is trehalose.

5. The method of claim 1, wherein said solution further comprises a potassium salt.

6. The method of claim 5, wherein said potassium salt is potassium phosphate.

7. The method of claim 1, wherein said solution further comprises α-crystallin.

8. The method of claim 1, wherein said solution further comprises a strong reducing agent.

9. The method of claim 8, wherein said strong reducing agent is ascorbic acid.

10. The method of claim 1, wherein said solution comprises a disaccharide, a-crystallin, ascorbic acid, and a potassium salt.

11. A method of claim 1, further wherein the loading is conducted at a temperature of between 25 and 40° C.

12. A method of claim 11, further wherein the loading is conducted at a temperature of between 30 and 40° C.

13. A method of claim 11, further wherein the loading is conducted at a temperature of about 37° C.

14. An erythrocyte loaded with from 10 mM to 50 mM trehalose.

15. An erythrocyte of claim 11, further comprising ascorbic acid.

16. An erythrocyte of claim 11, further comprising α-crystallin.

17. A method for separating fragile or damaged cells from a population of erythrocytes, said method comprising
contacting said population with a first solution which is hyperosmotic with respect to a solute,
loading a solute into said erythrocytes,
removing said erythrocytes from said hyperosmotic solution,
contacting said erythrocytes with a second solution which is mildly hypoosmotic in comparison to said hyperosmotic solution, thereby lysing fragile or damaged cells, and
separating said fragile or damaged cells from said population.

18. A method of claim 14, wherein said separation is by centrifugation.

19. A method for freeze-drying erythrocytes comprising lowering the hematocrit of said erythrocytes to between 2 and 5%.

20. A method for freeze-drying erythrocytes, comprising drying said erythrocytes in the presence of liposomes.

21. A method of claim 18, wherein said liposomes are composed primarily of unsaturated lipids.

22. A method for freeze-drying erythrocytes, comprising freeze-drying said erythrocytes in the presence of 200-300 mOsm of potassium salts.

23. A method of claim 19, wherein said erythrocytes are present in a hematocrit of up to 15%.

24. A buffer for drying erythrocytes, said buffer comprising liposomes.

25. A buffer of claim 21, wherein said liposomes are composed primarily of unsaturated lipids.

26. A buffer for drying erythrocytes, said buffer comprising ascorbic acid.

27. A buffer for rehydrating dried erythrocytes, said buffer comprising methylene blue.

28. A buffer for rehydrating dried erythrocytes, said buffer comprising transition metal ions.

29. A buffer of claim 24, wherein said transition metal ions are selected from the group consisting of zinc, copper, magnesium, and nickel.

30. A solution for rehydrating dried erythrocytes, said solution comprising ascorbic acid.

31. A solution for rehydrating dried erythrocytes, said solution comprising methylene blue, ascorbic acid, and transition metal ions.

32. A method for rehydrating dried erythrocytes, said method comprising contacting said dried erythrocytes with a solution comprising methylene blue.

33. A method for rehydrating dried erythrocytes, said method comprising contacting said dried erythrocytes with a solution comprising transition metal ions.

34. A method for rehydrating dried erythrocytes, said method comprising contacting said dried erythrocytes with a solution comprising ascorbic acid.

35. A method for rehydrating dried erythrocytes, said method comprising contacting said dried erythrocytes with a solution comprising methylene blue, and transition metal ions.
